(19) United States
(12) Patent Application Publication
(10) Pub. No.: US 2012/0231488 A1
(43) Pub. Date: Sep. 13, 2012

(54) FLUID SAMPLE COLLECTION DEVICE

(75) Inventors: Catriona Marshall, Valetta Way (GB); Anne Marie Crawford, Langley Heath (GB); Steven Andrew Ross, Hamstreet (GB); Timothy Joseph Nicholas Carter, Minster-on-Sea (GB)

(73) Assignee: VIVACTA LTD., Sittingbourne, Kent (GB)

(21) Appl. No.: 13/394,075

(22) PCT Filed: Sep. 1, 2010
(86) PCT No.: PCT/GB2010/051435

§ 371 Date: May 14, 2012

Related U.S. Application Data
(60) Provisional application No. 61/239,970, filed on Sep. 4, 2009.

(30) Foreign Application Priority Data
Sep. 3, 2009 (GB) 0915339.6

(51) Int. Cl. G01N 1/10; G01N 1/28; C12Q 1/02; G01N 33/50

(52) U.S. Cl. 435/29; 422/501; 422/68.1; 436/180

(57) ABSTRACT

Disclosed herein is a sample collection device for an aqueous fluid, such as whole blood, serum, plasma and urine. The device comprises a cartridge body defining an elongate sample collection passage that has open ends. The passage is arranged to draw the fluid into the passage by capillary action. The passage is provided along a portion of its length with a sample metering stop in the form of a hydrophobic coating arranged to prevent flow of the fluid by capillary action thereacross. A sample receiving portion of the passage extending between a collection end and the metering stop is non-linear, and preferably defines a pair of straight limbs connected by a bend. By providing a non-linear passage in this way, the maximum gravitational force which can act on the collected sample is reduced as compared to a conventional linear passage, thereby reducing the tendency of the sample to leak from the device and potentially avoiding the need for one or both ends of the passage to be sealed. The sample receiving portion of the passage may be provided with a hydrophilic coating to enhance the capillary action. A particularly suitable hydrophilic coating for a whole blood sample collection device is heparin, which may also serve as an anticoagulant for the blood.

### FIELD OF THE INVENTION

This present invention relates to a device for use in collecting and storing biological fluid samples such as whole (unseparated) blood, serum, plasma and urine taken from the human or animal body. Such samples may be used in diagnostic and other biochemical tests. More particularly, the present invention relates to such a device which relies on capillary action for the collection of the biological fluid sample.

The invention also relates to a test element comprising the fluid sample collection device.

### BACKGROUND TO THE INVENTION

Fluid samples taken from the human or animal body are required for a wide variety of diagnostic and other biochemical tests, including the measurement of immunological reactions (immunoassays). There is accordingly a need for a device which can be conveniently used for collecting and storing such samples. Since the samples may pose a microbiological contamination or heath risk, the device used for their collection should not allow unintended release of the samples during storage, transportation or manipulation. The sample collection device is preferably disposable.

A known sample collection device for whole blood comprises an open-ended linear capillary tube formed of glass. The tube typically has an internal diameter of between one and two millimetres. To prevent clotting of the collected blood, the internal surface of the tube may be coated with a suitable anticoagulant such as heparin, which may also serve to reduce the contact angle between the sample and the side of the tube.

In use of the known device, the skin on the tip of a patient's finger is pierced by a lancet or other sharp piercing member. The blood so elicited is drawn into the linear tube by capillary action. The volume of the blood sample and the rate at which it is collected may be maximised by holding the tube with a generally horizontal orientation. The volume of the sample collected in this way is usually less than 100 μL.

A problem associated with the blood sample collection device described above relates to the transportation and handling of the sample subsequent to its collection. In particular, when the orientation of the linear tube is changed, there is a risk that gravitational forces acting on the sample may exceed the intermolecular forces which maintain the sample in the tube, leading to the unintended release of a portion of the sample and the associated microbiological contamination or heath risk. This problem may be exacerbated when the linear tube is also subjected to accelerations caused by sudden movements or decelerations caused by small knocks, etc.

To prevent the unintended release of the sample, it is known to stopper one or both ends of the linear capillary tube, for example using silicone bungs or sealant. However, there remains a risk that a portion of the sample may be accidentally released before the ends of the tube have been sealed or after the seal has been removed for subsequent processing.

Thus, there is a need in the art for an improved sample collection device for biological fluids, which fluids are generally aqueous, and particularly such a device for which the risk of accidentally release of a portion of the sample subsequent to its collection may be reduced.

### SUMMARY OF THE INVENTION

According to an aspect of the present invention, there is provided a sample collection device for an aqueous fluid, the device comprising a body defining an elongate sample collection passage having open ends and arranged to draw the fluid into the passage by capillary action, wherein the passage is provided along a portion of its length with a sample metering stop arranged to prevent flow of the fluid by capillary action thereacross, and wherein a sample receiving portion of the passage extending between a collection end and the metering stop is non-linear.

By providing a non-linear passage in this way, the maximum gravitational forces which can act on the collected sample (with the device in any orientation) are reduced, as compared to a sample in a comparable linear capillary tube. The tendency of the sample to leak from the device may thereby be reduced, which may also avoid the need for one or both ends of the passage to be sealed after the sample has been collected.

The sample metering stop preferably comprises a portion of the surface of the passage that is hydrophobic. The hydrophobicity may be an inherent property of the material from which the body defining the passage is formed, or it may be provided by a coating applied to the surface of the passage, for example a waxy plastics material. The portion of the surface that is hydrophobic typically surrounds the passage and extends along a short portion of the length of the passage, for example about 5 mm. The metering stop serves not only to regulate the size of the collected sample, but also to control its position within the passage: the collected sample extends from the collection end of the passage to the edge of the sample metering stop.

Preferably, the surface of the sample receiving portion of the passage is hydrophilic. The hydrophilicity may be an inherent property of the material from which the body defining the passage is formed, or it may be provided by a coating applied to the surface of the passage. Suitable hydrophilic coating materials include proteins and sugars. A particularly preferred hydrophilic coating for a blood sample collection device comprises heparin, since this substance may also serve as an anticoagulant to prevent clotting of the blood sample. The heparin may be provided in a sugar matrix, which then forms a glassy amorphous coating for the passage. The hydrophilicity increases the intermolecular forces which draw the sample in the passage and maintain the sample in the passage. A high degree of hydrophilicity may also increase the speed of sample collection and/or facilitate the collection of larger sample volumes, since it allows a greater vertical height of fluid to be supported by intermolecular forces.

The sample receiving portion of the passage may have maximum and minimum transverse dimensions in the range 0.5 mm to 2.5 mm, preferably in the range 0.8 mm to 2.0 mm, more preferably 1.0 mm to 1.8 mm. The transverse dimensions are preferably substantially constant along the length of the sample receiving portion of the passage. Although dimensions in these ranges have been found to be generally suitable for collecting samples of most biological fluids, the appropriate dimensions for the sample receiving portion will depend to some degree on the physical properties of the fluid to be collected by the device and the required volume of the sample. Dimensions in excess of 5 mm have been found to be less suitable because gravity can then act on the meniscus to break up the sample within the device.

The sample receiving portion of the passage may have any cross-sectional shape, for example circular or substantially semi-circular (“U” shaped) cross-sections. A substantially semi-circular cross-sectional shape is particularly convenient if the passage is to be defined between two flat components in contact with each other, since only one of the components then needs to be grooved.

The volume of the sample receiving portion of the passage may be in the range 10 μL to 100 μL, preferably in the range 10 μL to 70 μL, and more preferably 20 μL to 50 μL.

According to the present invention, the sample receiving portion of the passage is non-linear. In this way, the maximum gravitational forces which can act on the collected sample (with the device in any orientation) are reduced, as compared to a sample in a conventional linear capillary tube of comparable type. Although such forces will be reduced for any non-linear shape, it is generally preferred that the sample receiving portion defines at least one bend, each bend defining an angle of about 90 degrees or about 180 degrees. The sum of the angles of the bends may exceed 180 degrees or 360 degrees.

In a particularly preferred embodiment of the present invention, the sample receiving portion of the passage defines at least two limbs which may be linear. The limbs may be parallel and/or laterally adjacent to each other. The adjacent limbs may be linked by at least one bend, such as a 180 degree bend. Each of the limbs of the sample receiving portion may have a length in the range 10 mm to 50 mm, and preferably in the range 15 mm to 40 mm. With this arrangement, and with the limbs in a substantially vertical orientation, the hydrostatic pressure in the collected sample can be at least partially balanced, thereby reducing the height of the sample which needs to be supported by intermolecular forces.

In some embodiments, the sample collection passage defines a single plane. In this way, the thickness of the device can be minimised. The device may have a substantially rectangular, plate-like form. The collection end of the passage may be provided at or in the vicinity of a corner of the device, to thereby allow for convenient manipulation and handling of the device during collection of the fluid sample.

The body defining the sample collection passage may be formed of a plastics material, such as polymethyl methacrylate (acrylic). The body may be a moulded component and may be at least partially transparent so that the flow of the fluid in the sample receiving portion of the passage can be observed.

According to another aspect of the invention, there is provided a test element comprising the sample collection device described hereinabove, wherein the body defining the sample collection passage further defines at least one analytical chamber in fluid communication with a delivery end of the sample collection passage, that is to say an end of the passage other than the collection end. Each analytical chamber may be provided with an analytical reagent. The body may further define a port in fluid communication with the at least one analytical chamber. In use of the device, the port may be connected to a vacuum source to draw a collected sample over the hydrophobic metering stop and into the at least one analytical chamber. Alternatively, a positive pressure source may be connected to the collection end of the sample collection passage.

In a particularly preferred embodiment of the present invention, the test element, including the sample collection device, the at least one analytical chamber and the analytical reagent, is in the form of a cartridge. The cartridge may further comprise a sensor or transducer in the vicinity of the at least one analytical chamber together with exposed electrical connectors coupled to the sensor or transducer. The portion of the body defining the at least one analytical chamber may be transparent to enable its content to be irradiated with electromagnetic radiation.

The cartridge may be arranged for use with a test apparatus, which apparatus is arranged to receive the cartridge, draw the collected sample into the at least one analytical chamber, irradiate the content of the at least one analytical chamber with electromagnetic radiation and read an electrical signal received from the sensor or transducer via the electrical connectors. The test apparatus may comprise a processing means arranged to provide a result of the analysis to a display.

The cartridge may be designed for a single use, after which use it can be disposed of, thereby avoiding the need for cleaning and/or sterilisation.

According to another aspect of the present invention, there is provided use of a sample collection device for collecting an aqueous fluid sample, the device comprising a body defining an elongate sample collection passage having open ends and arranged to draw the fluid into the passage by capillary action, wherein the passage is provided along a portion of its length with a sample metering stop arranged to prevent flow of the fluid by capillary action thereacross, and wherein a sample receiving portion of the passage extending between a collection end and the metering stop is non-linear.

In a preferred embodiment, the aqueous fluid is a biological fluid, such as whole blood, serum, plasma or urine.

Further features and advantages will be apparent from the detailed description of the present invention provided hereinbelow.

### BRIEF DESCRIPTION OF THE DRAWINGS

Specific embodiments of the present invention will now be described with reference to the accompanying drawings, in which:

FIG. 1 is a schematic view of a known analytical system;

FIG. 2 illustrates a test apparatus and test element embodying the known system shown in FIG. 1;

FIG. 3 is a perspective view of a test element comprising a sample collection device according to the invention;

FIG. 4 is a more detailed view of the test element shown in FIG. 3, with components removed to show the sample collection device;

FIG. 5 shows the sample collection device shown in FIG. 4 in more detail; and

FIGS. 6a to 6d are schematic views of the sample collection device shown in FIG. 4 for use in explaining its behaviour.

### DETAILED DESCRIPTION

As used herein, a hydrophobic surface is generally understood to be one with which a droplet of water defines a static contact angle of greater than 90 degrees. Similarly, as used herein, a hydrophilic surface is generally understood to be one with which a droplet of water defines a static contact angle of less than 90 degrees. Static contact angles may be measured using a contact angle goniometer.

The present invention provides a sample collection device for an aqueous fluid, such as whole blood, serum, plasma or urine. The device comprises a body defining an elongate sample collection passage having open ends and arranged to draw the fluid into the passage by capillary action. According to the present invention, the passage is provided along a portion of its length with a sample metering stop arranged to prevent flow of the fluid by capillary action thereacross. A sample receiving portion of the passage extending between a collection end and the metering stop is non-linear. By providing a non-linear sample receiving portion, the risk of the sample leaking from the device may be reduced.

The present invention thus provides a convenient means for collecting a biological fluid sample for use in a wide variety of diagnostic and other biochemical tests, including the measurement of immunological reactions (immunoassays). Known systems for carrying out such tests, to which the invention may be applied, are disclosed in U.S. Pat. No. 5,622,868 and WO 2004/090512 A1 and will be described hereinbelow.

The known systems employ reagents which undergo a detectable colour change or cause migration of a coloured species to provide an indication of an analyte in a biological fluid sample. The systems may be arranged to detect the colour change by irradiating the reagent with electromagnetic radiation of an appropriate wavelength, and then detecting absorption of the radiation as microscopic heating of a pyroelectric transducer arranged in the vicinity of the reagent. Migration of a colour species can be detected by analysing the time delay between the pulsed irradiation of a sample with electromagnetic radiation and the subsequent microscopic heating of the transducer caused by absorption of the radiation by the coloured species.

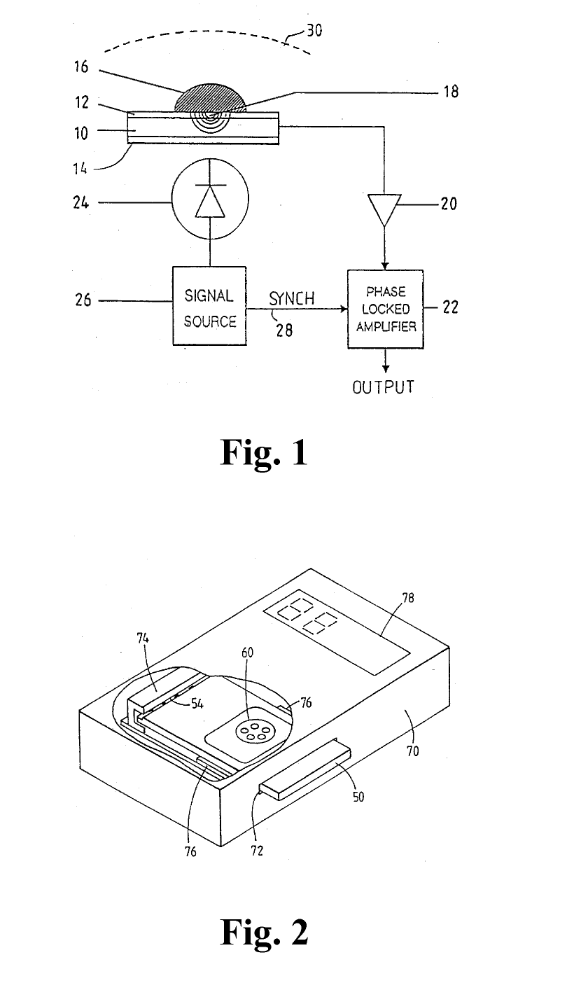

FIG. 1 is a schematic view of a known system, which includes a test element carrying the pyroelectric transducer. Referring to the figure, the transducer includes a polyvinylidene fluoride (PVDF) film 10 having electrode coatings 12, 14 on the upper and lower surfaces respectively. The electrode coatings are formed of indium tin oxide (ITO) having a thickness in the range 5 nm to 100 nm. Strips of reagent 16 are deposited, using any suitable technique, upon the upper electrode coating 12 of the transducer.

The electrode coatings 12 and 14 are connected, via exposed electrical connectors on the test element, to a test apparatus. The connectors (not shown) are coupled to the inputs of a charge amplifier 20 presenting a high input impedance, and the output of the charge amplifier is taken to a phase locked amplifier 22. A light source 24 of the test apparatus, in the form of a light emitting diode (LED), is positioned so as to illuminate the reagent strips through the pyroelectric film 10 and its associated electrode coatings. The light source is powered through a modulator 26 which provides a square wave output typically up to about 15 Hz. A reference signal is taken on line 28 from the modulator 26 to the phase locked amplifier 22.

In use of the known system, a biological fluid sample is collected in a known manner and deposited upon the surface of the pyroelectric transducer of the cartridge, the outline of the drop being shown in the drawing at 30. In the presence of levels of the analyte, the appropriately chosen reagent undergoes a change in optical absorption. Light of an appropriate wavelength from the source 24 is absorbed in the reagent, causing microscopic heating over a localised region 18. This heating is sensed by the transducer and results in a change in output from the amplifier 20. Through phase locking on the reference signal on line 28, the amplifier 22 is able to provide a sensitive output signal indicative of the heating and thus of the light absorption within the reagent and presence of the analyte within the biological fluid sample. The output of phase locked amplifier 22 is digitised and sent on an appropriate bus to a microprocessor.

The type of reagent chosen will vary widely depending upon the analytical procedure. For example, in tests for ions, pH and heavy metal indicator dyes may be employed which change colour on chelation/binding of ions. A variety of reagents are known for assays of metabolites, drugs and biochemicals in blood and urine. One example is a paracetamol assay with production of aminophenol from paracetamol by arylacylamidase. In immunological assays, the reagent may take the form of a protein or microbial antigen. The reagent may also be the antibody. The technique is also applicable to enzyme linked immunosorbent assays (ELISA).

FIG. 2 illustrates a test apparatus 70 and test element 50 embodying the known system shown in FIG. 1. The test element 50 is in the form of a single use cartridge, thereby removing the problems of contamination and of cleaning potentially hazardous sample material. The test element 50 comprises an inert transparent substrate of rectangular form. At one end, the substrate is provided with electrical connectors 54 enabling the test element to be plugged into the test apparatus 70. The substrate carries the pyroelectric transducer comprising the PVDF film 10 having electrode coatings 12, 14. A well 60 is defined over the transducer for receiving the biological fluid sample.

With further reference to FIG. 2, the test apparatus 70 comprises a housing provided with a slot 72 into which the test element 50 can be slidingly engaged. Internally, the housing provides an edge connector 74 designed to mate with the electrical connectors 54 on the test element 50. A light source shown schematically at 76 is positioned within the housing 70 so as to be aligned with the well 60 when the test element 50 is fully engaged.

The test apparatus 70 contains circuitry (not shown) providing the modulated signal source, charge amplifier and phase locked amplifier as described hereinabove with reference to FIG. 1. There is further provided a microprocessor, which may be of commercially available form, which is connected to receive the output of the phase locked amplifier and to control a display 78.

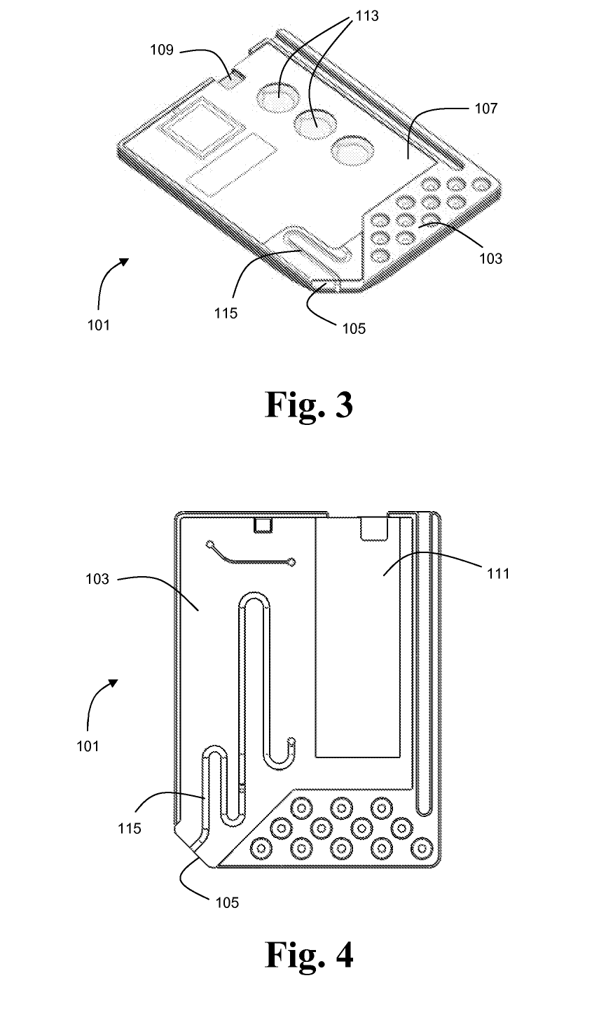

The present invention provides a fluid sample collection device which may be integrated into the test element 50 described hereinabove with reference to FIGS. 1 and 2. A test element including the sample collection device according to the invention is shown in FIG. 3. The test element is also shown in FIG. 4 with some components removed for clarity.

With reference to FIGS. 3 and 4, the test element according to the invention is a single use cartridge 101 arranged for use with a test apparatus similar to that shown in FIG. 2. The cartridge 101 comprises a thin-walled transparent main body 103 moulded from polymethyl methacrylate (acrylic). The main body 103 has a substantially rectangular shape, with one of its four corners being provided with a chamfer 105. The cartridge 101 also comprises a number of thin layers 107 which, together with the main body 103, form a laminate providing various structures, which are described hereinbelow.

The cartridge 101 is provided with electrical connectors 109 arranged along the edge of the main body, which connectors enable the cartridge 101 to be plugged into the test apparatus (not shown). The cartridge 101 also carries a pyroelectric transducer 111 of the type described hereinabove with reference to FIGS. 1 and 2. A plurality of analytical chambers 113 are arranged over the transducer 111. Appropriate reagents are provided in the chambers 113 in the vicinity of an upper surface of the transducer 111.

The cartridge also comprises a sample collection device 115 in the form of an elongate sample collection passage moulded into the main body 103. The collection device 115 is arranged to draw a fluid sample, which in this case is whole (unseparated) blood, into the collection passage by capillary action.

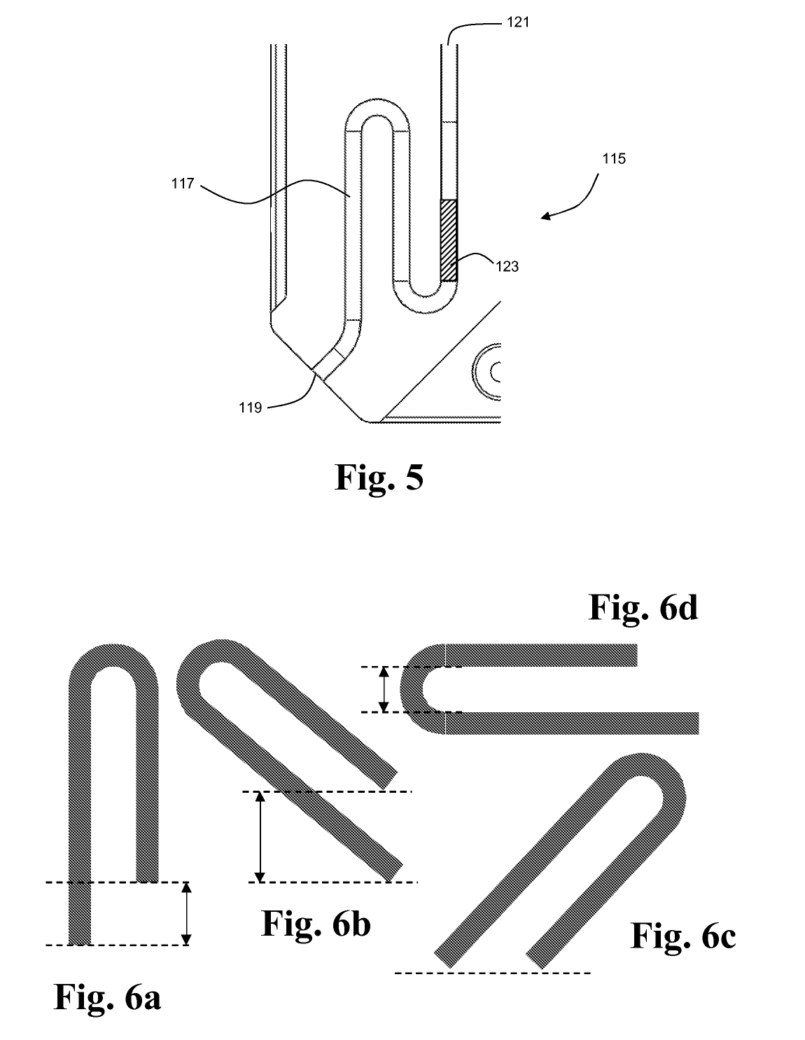

The sample collection device 115 is shown in more detail in FIG. 5. With reference to this figure, the elongate sample collection passage 117 extends from a collection end 119, which terminates at the corner of the main body 103 provided with the chamfer 105, to a delivery end 121, which terminates within the cartridge 101 and is in fluid communication with the analytical chambers 113. Both ends of the collection passage 117 are also in fluid communication with the ambient atmosphere—that is to say they are not sealed.

The cross-sectional shape of the sample collection passage 117 is substantially semi-circular (being formed from a channelled plate which is closed by a flat lid). The passage has a width of 1.15 mm.

An intermediate portion of the sample collection passage 117 is provided with a sample metering stop 123 arranged to prevent flow of the collected sample by capillary action thereacross. The sample metering stop 123 comprises a portion of the surface of the sample collection passage 117 which is coated with a hydrophobic wax, which wax is preferably non-water soluble. The hydrophobic wax at least partially surrounds the sample collection passage 117 and is coated along approximately 5 mm of the length of the passage 117.

A sample receiving portion of the passage 117 is defined between the collection end 119 of the passage and the metering stop 123. The surface of the sample receiving portion is provided with a hydrophilic coating in the form of heparin, which also serves as an anticoagulant for the collected blood sample. The hydrophilic coating reduces the contact angle between the sample and the surface of the passage 117, and therefore enhances the capillary effect which draws the sample into the device 115.

The volume of the sample receiving portion defined between the collection end 119 and the metering stop 123 is approximately 30 μL.

According to the invention, the sample receiving portion of the passage 117 is non-linear. In the embodiment shown in FIG. 5, the sample receiving portion is substantially U-shaped, with a pair of substantially parallel limbs being connected by a bend defining an angle of about 180 degrees. As shown in the figure, the limb adjacent to the collection end 119 of the passage 117 is longer than the limb adjacent to the sample metering stop 123. The particular shape of the sample receiving portion according to the invention reduces the maximum gravitational forces on the collected sample (with the device in any orientation), thereby reducing the risk of the blood sample leaking from the device 115.

In use of the cartridge 101, the skin on the tip of a patient's finger is pierced and the collection end 119 of the sample collection device 115 is presented to the blood so elicited. With the sample collection passage 117 in a substantially horizontal orientation the blood is drawn into the sample receiving portion of the passage 117 by capillary action. The collected blood sample is metered by the sample metering stop 123, which prevents flow of the blood by capillary action thereacross. The sample metering stop 123 also serves to control the position of the collected blood sample within the passage 117.

To perform an analysis, the cartridge 101 containing the collected blood sample is inserted into the testing apparatus. The blood sample is drawn across the sample metering stop 123 by applying a vacuum source to a port of the main body 103 in fluid communication with the analytical chambers 113. The blood sample is drawn into the analytical chambers 113 whereupon it comes into contact with the reagents, which undergo a detectable colour change or cause migration of a coloured species towards the surface of the pyroelectric transducer 111 to provide an indication of an analyte in a biological fluid sample. The colour change may be detected by irradiating the reagent with electromagnetic radiation of an appropriate wavelength, and then detecting absorption of the radiation as microscopic heating of the pyroelectric transducer 111. Migration of a colour species can be detected by analysing the time delay between the pulsed irradiation of a sample with electromagnetic radiation and the subsequent microscopic heating of the transducer 111 caused by absorption of the radiation by the coloured species.

After collection of the blood sample, the cartridge 101 may need to be stored or transported before it can be used with the testing apparatus. Even when storage or transportation is not required, some manipulation of the cartridge 101 will generally be required before it can be inserted into the testing apparatus. This manipulation, which may for example include changing the orientation of the cartridge 101, may result in the gravitational forces acting on the blood sample exceeding the intermolecular forces which maintain the sample in the passage 117. However, the particular shape of the sample receiving portion as described hereinabove reduces the maximum gravitational forces on the collected sample, thereby reducing the risk of the blood sample leaking from the device 115. In this way, the need for the collection end 119 of the sample collection passage 117 to be sealed may be avoided and the security of the blood sample is generally enhanced.

The behaviour of the sample collected in the device 115 will now be explained in more detail with reference to FIGS. 6a to 6d, which are schematic views of the device 115 in different orientations. As described hereinabove, the sample receiving portion of the device 115 comprises a pair of substantially parallel limbs connected by a bend defining an angle of about 180 degrees.

In any orientation of the sample collection device 115, it is preferred that the vertical height of the sample that needs to be supported solely by capillary action is significantly less than the maximum height of the sample that can be supported by capillary action. It is particularly preferred that the vertical height that needs to be supported is less than 50%, or less than 20%, of the maximum height that can be supported.

The maximum height of a sample that can be supported by capillary action in a passage having a circular cross-section is given by the following equation:

$$h = \frac{2 γcos θ}{ρ gr} \qquad ( 1 )$$

where h is the vertical height of the sample that can be supported, γ is the surface tension between the sample and air, θ is the contact angle between the sample and the side of the tube, ρ is the density of the sample, g is acceleration due to gravity and r is the radius of the sample collection passage 117.

The vertical height of the sample that needs to be supported is illustrated in each of FIGS. 6a to 6d with a double headed arrow. In the orientations shown in FIGS. 6a and 6b, the height which needs to be supported is reduced by balancing the hydrostatic pressure in each of the limbs of the sample receiving portion. In the orientation shown in FIG. 6c, the hydrostatic pressures are completely balanced, so that there is no sample height which needs to be supported by capillary action. In the orientation shown in FIG. 6d, there is no balancing of hydrostatic pressures, but the height which needs to be supported is relatively small.

It will be appreciated that the maximum vertical height of the sample which needs to be supported compares favourably with that of a sample contained in a linear collection passage.

Although the present invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

For example, in the embodiment described above the fluid collection device is incorporated into a cartridge having analytical chambers and a transducer. The fluid collection device may, however, be provided on its own, for use with a separate testing apparatus. Alternatively, the fluid collection device may be incorporated into other devices for which a sample of an aqueous fluid is required.

The U-shaped sample receiving portion of the embodiment described above is purely exemplary. In other embodiments the sample receiving portion may have any non-linear shape. For example, the sample receiving portion may comprise a series of more than two parallel limbs connected by bends. Alternatively, part of the sample receiving portion could be spiral-shaped. Sample receiving portions having three-dimensional shapes are also possible. In general, it is preferred that the sample receiving portion is shaped to reduce the maximum gravitational forces that can act on a collected sample.

1. A sample collection device for an aqueous fluid, the device comprising a body defining an elongate sample collection passage having open ends and arranged to draw the fluid into the passage by capillary action, wherein the passage is provided along a portion of its length with a sample metering stop arranged to prevent flow of the fluid by capillary action thereacross, and wherein a sample receiving portion of the passage extending between a collection end and the metering stop is non-linear.

2. A sample collection device as claimed in claim 1, wherein the sample metering stop comprises a portion of the surface of the passage that is hydrophobic.

3. A sample collection device as claimed in claim 1, wherein the surface of the sample receiving portion of the passage is hydrophilic.

4. A sample collection device as claimed in claim 3, wherein the surface of the sample receiving portion of the passage is provided with a hydrophilic coating.

5. A sample collection device as claimed in claim 4, for whole blood, wherein the hydrophilic coating comprises at least one selected from heparin, a protein and a sugar.

6. A sample collection device as claimed in claim 1, wherein the sample receiving portion of the passage has maximum and minimum transverse dimensions in the range 0.5 mm to 2.5 mm.

7. A sample collection device as claimed in claim 6, wherein the cross-sectional shape of the sample collection passage is substantially semi-circular.

8. A sample collection device as claimed in claim 1, wherein the volume of the sample receiving portion of the passage is in the range 10 μL to 100 μL.

9. A sample collection device as claimed in claim 1, wherein the sample receiving portion of the passage defines at least one bend, each bend defining an angle of at least 90 degrees.

10. A sample collection device as claimed in claim 1, wherein the sample receiving portion of the passage defines at least one bend, the sum of the angles of the bends exceeding 180 degrees.

11. A sample collection device as claimed in claim 1, wherein the sample receiving portion of the passage defines at least two linear limbs, adjacent pairs of the limbs being linked by at least one bend.

12. A sample collection device as claimed in claim 11, wherein each of the straight limbs of the sample receiving portion of the passage has a length in the range 10 mm to 50 mm.

13. A sample collection device as claimed in claim 11, wherein the straight limbs are parallel to each other.

14. A sample collection device as claimed in claim 1, wherein the sample collection passage extends in a single plane.

15. A sample collection device as claimed in claim 1, wherein the body defining the sample collection passage is formed of a plastics material.

16. A sample collection device as claimed in claim 1, wherein the body defining the sample collection passage is transparent at least in the region of the sample receiving passage, such that a user can see the flow of the fluid in the passage.

17. A test element comprising the sample collection device for an aqueous fluid, the device comprising a body defining an elongate sample collection passage having open ends and arranged to draw the fluid into the passage by capillary action, wherein the passage is provided along a portion of its length with a sample metering stop arranged to prevent flow of the fluid by capillary action thereacross, and wherein a sample receiving portion of the passage extending between a collection end and the metering stop is non-linear, wherein the body defining the sample collection passage further defines at least one analytical chamber in fluid communication with a delivery end of the sample collection passage.

18. A test element as claimed in claim 17, wherein each of the analytical chambers is provided with an analytical reagent.

19. A test element according to claim 17, wherein the body defining the sample collection passage further defines a port in fluid communication with the at least one analytical chamber, the port being arranged for connection to a vacuum source for drawing a collected sample over the sample metering stop and into the at least one analytical chamber.

20. A sample collection device according to claim 1, wherein the body defining the sample collection passage has a rectangular plate-like shape.

21. A method of using a sample collection device according to claim 1 for collecting an aqueous fluid sample.

22. A method of using a sample collection device according to claim 1 for collecting whole blood, serum, plasma or urine.

23. A test element according to claim 17, wherein the body defining the sample collection passage has a rectangular plate-like shape.
